(19) United States
(12) Patent Application Publication
(10) Pub. No.: US 2012/0283449 A1
(43) Pub. Date: Nov. 8, 2012

(54) METHOD FOR CARRYING OUT SEQUENTIAL REACTIONS USING A HEATING MEDIUM HEATED BY MEANS OF INDUCTION

(76) Inventors: Carsten Friese, Duesseldorf (DE); Andreas Kirschning, Celle (DE); Sascha Ceylan, Hannover (DE); Jens Wegner, Hannover (DE)

(21) Appl. No.: 13/444,922

(22) Filed: Apr. 12, 2012

Related U.S. Application Data
(63) Continuation of application No. PCT/EP2010/059213, filed on Jun. 29, 2010.

(30) Foreign Application Priority Data
Oct. 13, 2009 (DE) 102009045636.8

(51) Int. Cl. C07D 333/24

(52) U.S. Cl. 549/79

(57) ABSTRACT

A method for performing at least two successive chemical reactions to produce a target compound from at least one first, and at least one second and/or a further reactant, comprising a first reaction and a further reaction in a reaction medium, the reaction medium in contact with a solid heating medium heated by electromagnetic induction and which is located inside the reactor and surrounded by the reaction medium, and the heating medium being heated by electromagnetic induction with the aid of an inductor, an intermediate compound being formed in a first reaction, from which the target compound forms in a further reaction, and at least one reactant, not present in the reactor before the first reaction, being added between the first and the further reaction, and the target compound being separated from the heating medium.

### CROSS-REFERENCE TO RELATED APPLICATIONS

This application is a continuation of International Patent Application No. PCT/EP2010/059213 filed Jun. 29, 2010, which claims priority to German Patent Application No. 102009045636.8 filed Oct. 13, 2009, the contents of both of which are incorporated herein by reference.

The present invention lies within the field of chemical synthesis and relates to a method for performing sequential, in other words successively proceeding, chemical reactions using an inductively heated heating medium.

Various methods are known for heating the reactants in order to perform chemical reactions that can be induced thermally. The most widespread of these is heating by means of heat conduction. Here the reactants are located in a reactor, and either the walls of the reactor itself are heated or heat-transmitting elements such as heating coils or heat exchanger tubes or plates, for example, are built into the reactor. This method is comparatively sluggish, such that on the one hand heating of the reactants occurs slowly and on the other the input of heat cannot be stopped quickly or even replaced by cooling. An alternative consists of heating the reactants by radiating microwaves into the reactants themselves or into a medium containing the reactants. Microwave generators present a considerable risk to safety, however, as they use complex equipment and there is a danger of an escape of radiation.

By contrast, the present invention provides a method in which the reaction medium is heated by bringing it into contact with a heating medium that can be heated by electromagnetic induction, said heating medium being heated “from outside” by electromagnetic induction with the aid of an inductor.

The inductive heating method has been used in industry for some time. The commonest applications are melting, hardening, sintering and the heat treatment of alloys. However, processes such as gluing, shrinking or joining of components are also known applications of this heating method.

Methods for isolating and analyzing biomolecules are known from the German patent application DE 198 00 294, wherein said biomolecules are bound to the surface of magnetic particles that can be heated by induction. This document states:

“The principle of operation consists in adsorptively or covalently binding biomolecules to the surface of a functional polymer matrix, in which the inductively heatable magnetic colloids or finely dispersed magnetic particles are encapsulated, said biomolecules being capable of binding analytes such as e.g. DNA/RNA sequences, antibodies, antigens, proteins, cells, bacteria, viruses or fungal spores according to the complementary affinity principle. Once the analytes have been bound to the matrix the magnetic particles can be heated in a high-frequency magnetic alternating field to temperatures of preferably 40 to 120° C. that are relevant for analysis, diagnostics and therapy.” This document goes on to cover the technical design of coil systems and high-frequency generators that can be used in this method. The cited document thus describes the use of inductively heatable particles in the analysis of complex biological systems or biomolecules.

DE 10 2005 051637 describes a reactor system with a microstructured reactor and methods for performing a chemical reaction in such a reactor. The reactor as such is heated by electromagnetic induction. Heat is transferred into the reaction medium via the heated reactor walls. This limits the size of the surface area that is available for heating the reaction medium. It is also necessary to heat parts of the reactor that are not in direct contact with the reaction medium.

U.S. Pat. No. 5,110,996 describes the production of vinylidene fluoride by reacting dichlorodifluoromethane with methane in the gas phase in a heated reactor. The reactor is filled with a non-metallic filler. The reaction chamber containing this filler is encased by a metallic jacket, which is heated from outside by electromagnetic induction. Thus the reaction chamber itself is heated from outside, causing the filler likewise to be heated at the same time by heat radiation and/or heat conduction. Direct heating by electromagnetic induction of the filler around which the reactants flow does not occur even if this filler is electrically conductive, as the metallic reactor wall shields the electromagnetic fields from the induction coil.

WO 95/21126 discloses a method for producing hydrogen cyanide in the gas phase from ammonia and a hydrocarbon using a metallic catalyst. The catalyst is located inside the reaction chamber, such that the reactants flow around it. It is heated from outside by electromagnetic induction at a frequency of 0.5 to 30 MHz, in other words by an alternating field in the high-frequency range. This document quotes the aforementioned document U.S. Pat. No. 5,110,996, with the comment that inductive heating is conventionally performed in the frequency range from approximately 0.1 to 0.2 MHz. This statement is not however included in the cited U.S. Pat. No. 5,110,996, so it is unclear to what it relates.

WO 00/38831 addresses controlled adsorption and desorption processes, in which the temperature of the adsorbent material is controlled by electromagnetic induction.

It is known from the journal article “Induktives Heizen in der organischen Synthese . . . ” by S. Ceylan, C. Friese, Ch. Lammel, K. Mazac and A. Kirschning, Angew. Chem. 2008 (129), pp. 9083-9086, Angew. Chem. Int. Ed. 2008 (47), pp. 8950-8953, that chemical reactions can be performed by heating a heating medium using electromagnetic induction. The German patent application DE 102007059967 and the world patent application WO 2009/074373 have since been published, which describe the subject-matter of the cited journal article in more detail. A number of reactions are mentioned by way of example. They do not include successive chemical reactions in which a target compound forms via at least one intermediate compound following addition of a further reactant.

The present invention provides in a general embodiment a method for performing at least two successive chemical reactions to produce a target compound from at least one first, and at least one second and/or further reactant, encompassing a first reaction and a further reaction in a reactor containing a reaction medium, the reaction medium in the reactor being brought into contact with a solid heating medium that can be heated by electromagnetic induction and that is located inside the reactor and surrounded by the reaction medium, and the heating medium being heated by electromagnetic induction with the aid of an inductor, an intermediate compound being formed in a first reaction, from which the target compound forms in a further reaction, and at least one reactant being added to the reactor between the first and the further reaction which was not present in the reactor before the first reaction and the target compound being separated from the heating medium.

The target compound can form by way of example but in a non-limiting manner through one of the following reaction sequences:
a) within the first reactant an intramolecular bond rearrangement or a fragmentation takes place as a first reaction to form an intermediate compound, which is reacted with one or more second reactants in a further reaction to form the target compound;
b) the first reactant reacts in a first reaction with a second reactant to form an intermediate compound, from which the target compound forms in a further reaction with a further reactant;
c) the first reactant reacts in a first reaction with a second reactant to form a first intermediate compound, from which in a second reaction a second intermediate compound forms through an intramolecular bond rearrangement or a fragmentation, from which the target compound forms in a further reaction with a further reactant.

A “further reactant” is therefore understood to be a reactant that reacts with an intermediate compound. The first and the further reaction can take place at separate times in the same reactor, wherein after the intermediate compound has at least largely formed (for example up to close to the equilibrium state) a further reactant is added in order to obtain the target compound from the intermediate compound in a further reaction.

It can be convenient for the first and the further reaction to be separated not only in time but also in space, in other words for them to proceed in different reactors. As is stated below, continuous-flow reactors arranged in series are preferably used in this case. A preferred embodiment of the present invention thus consists of a method for performing at least two successive chemical reactions to produce a target compound from at least one first and at least one second and/or a further reactant, the first reaction taking place in a first reactor and the further reaction taking place in a second reactor, each containing a reaction medium, the reaction medium in each reactor being brought into contact with a solid heating medium which can be heated by electromagnetic induction and which is located inside the reactor and surrounded by the reaction medium, and the heating medium being heated by electromagnetic induction with the aid of an inductor, at least one reactant being added to the second reactor which was not present in the first reactor.

Additional reaction steps are not excluded here.

The “target compound” is understood to be any compound obtained in macroscopic amounts as a result of the method according to the invention as an isolated substance, as a component of a mixture of substances or as a solution in a solvent. Macroscopic amounts are understood to be amounts of at least 100 mg, preferably at least 1 g and in particular at least 100 g per working day. By contrast, the intermediate compound is not isolated as such but is rather reacted in one or more further reaction steps to form the target compound. However, an intermediate compound is distinguished from a “transitional state” of a chemical reaction by the fact that it is sufficiently stable to be able to be isolated or at least detected by spectroscopic methods (e.g. IR or Raman spectrum, UV/visible spectrum, NMR or ESR spectrum).

The chemical reactions are therefore started and optionally maintained by heating a reaction medium containing the corresponding reactants. This includes the possibility that the reaction medium, a liquid for example, is itself involved in the reaction and is hence a reactant. A reactant can be dissolved or dispersed in the reaction medium, wherein the reaction medium itself can be inert or can itself be a reactant. Alternatively, one, two or more reactants are present in dissolved or dispersed form in a reaction medium that is itself not changed by the chemical reactions.

The solid heating medium is surrounded by the reaction medium. This can mean that the solid heating medium, with the exclusion of possible edge zones, is located within the reaction medium, for example if the heating medium is in the form of particles, chips, wires, meshes, wool, fillers, etc. However, it can also mean that the reaction medium flows through the heating medium through a large number of holes in the heating medium if the latter consists for example of one or more membranes, a bundle of tubes, a rolled metal foil, frits, porous fillers or a foam. In this case too the heating medium is substantially surrounded by the reaction medium, as the majority of its surface area (90% or more) is in contact with the reaction medium. By contrast, in a reactor whose outer wall is heated by electromagnetic induction (in accordance with the cited document U.S. Pat. No. 5,110,996 for example), only the inner surface of the reactor is in contact with the reaction medium.

The wall of the reactor is made from a material that does not shield or absorb the electromagnetic alternating field generated by the inductor and so does not heat up itself. Metals are therefore unsuitable. It can consist for example of plastic, glass or ceramic (such as for example silicon carbide or silicon nitride). The latter is suitable in particular for reactions at high temperature (500 to 600° C.) and/or under pressure. A special reactor material, which can also be used for reactions under moderate pressure (up to approximately 10 bar) is polyetheretherketone (PEEK).

The processing mode described above has the advantage that the heat energy for triggering and/or performing the chemical reactions is not introduced into the reaction medium via surfaces such as for example the reactor walls, heating coils, heat exchanger plates or similar but rather it is generated directly in the body of the reactor. The ratio of heated surface area to volume of the reaction medium can be substantially greater than in the case of heating via heat-transferring surfaces, as is also the case for example in accordance with DE 10 2005 051637 cited in the introduction. In addition, the efficiency of electric current to heating capacity is improved. By switching on the inductor, heat can be generated in the entire solid heating medium, which is in contact with the reaction medium via a very large surface area. When the inductor is switched off, the further introduction of heat is very quickly stopped. This allows a very targeted reaction control.

Once the target compound has been formed, it is separated from the heating medium. Ideally the target compound is isolated in pure form, in other words without solvent (which can be separated by distilling off the solvent or by precipitating the target compound from the solvent) and with no more than the usual impurities. However, the target compound can also be separated from the heating medium in a mixture with reactants or as a solution in the reaction medium and only subsequently isolated by further processing or transferred to a different solvent if desired. The method can therefore be used for the preparative production of the target compound to enable it to be used further.

By contrast, there are methods in which a chemical reaction is likewise started by electromagnetic induction of a heating medium but this reaction is not used to produce a target compound that is separated from the heating medium after the end of the reaction. One example of this is the curing of resin systems, in which the curing reaction is started at particles that are dispersed in the resin system and are heated by electromagnetic induction. These particles remain in the cured resin system and no defined target compound is isolated. The same applies to the converse situation in which an adhesive bond is dissolved again by the inductive heating of particles in the adhesive matrix. A chemical reaction can take place here, but no target compounds are isolated.

The heating medium consists of an electrically conductive and/or magnetizable material that heats up under the influence of an electromagnetic alternating field. It is preferably selected from materials having a very large surface area in comparison to their volume. For example, the heating medium can be selected from electrically conductive chips, wires, meshes, wool, membranes, porous frits, tube bundles (comprising three or more tubes), rolled metal foil, foams, fillers such as for example granules or balls, Raschig rings and in particular from particles, which preferably have an average diameter of no more than 1 mm. For example, metallic mixing elements such as are used for static mixers can be used as the heating medium. In order to be capable of being heated by electromagnetic induction, the heating medium is electrically conductive, for example metallic (wherein it can be diamagnetic), or it has an increased interaction with a magnetic field in comparison to diamagnetism and is in particular ferromagnetic, ferrimagnetic, paramagnetic or superparamagnetic. It makes no difference whether the heating medium is organic or inorganic in nature or whether it contains both inorganic and organic components.

In a preferred embodiment the heating medium is selected from particles of electrically conductive and/or magnetizable solids, wherein the particles have an average particle size in the range from 1 to 1000, in particular from 10 to 500 nm. The average particle size and if necessary also the particle size distribution can be determined by light scattering, for example. Magnetic particles, for example ferromagnetic or superparamagnetic particles, are preferably chosen that have as low as possible a remanence or residual magnetization. This has the advantage that the particles do not adhere to one another. The magnetic particles can be present for example in the form of “ferrofluids”, in other words liquids in which ferromagnetic particles are dispersed in the nano-size scale. The liquid phase of the ferrofluid can then serve as the reaction medium.

Magnetizable particles, in particular ferromagnetic particles, having the desired properties are known in the prior art and are commercially available. The commercially available ferrofluids are mentioned by way of example. Examples of the production of magnetic nanoparticles that can be used in the context of the method according to the invention can be taken from the article by Lu, Salabas and Schüth: “Magnetische nano-Partikel: Synthese, Stabilisierung, Funktionalisierung and Anwendung”, Angew. Chem. 2007, 119, pages 1242 to 1266.

Suitable magnetic nanoparticles are known with differing compositions and phases. Examples that can be cited include: pure metals such as Fe, Co and Ni, oxides such as Fe₃O₄ and gamma-Fe₂O₃, spinel-like ferromagnets such as MgFe₂O₄, MnFe₂O₄ and CoFe₂O₄ and alloys such as CoPt₃ and FePt. The magnetic nanoparticles can have a homogeneous structure or a core-shell structure. In the latter case the core and shell can consist of different ferromagnetic or antiferromagnetic materials. Embodiments are also possible, however, in which at least one magnetizable core, which can be ferromagnetic, antiferromagnetic, paramagnetic or superparamagnetic for example, is surrounded by a non-magnetic material. This material can be an organic polymer, for example. Alternatively the shell consists of an inorganic material such as for example silicic acid or SiO₂. A coating of this type can prevent a chemical interaction between the reaction medium or reactants and the material of the magnetic particles themselves. Furthermore, the shell material can be surface-functionalized without the material of the magnetizable core interacting with the functionalizing species. Several particles of the core material can also be enclosed together in such a shell.

Nanoscale particles of superparamagnetic substances selected from aluminum, cobalt, iron, nickel or alloys thereof, metal oxides of the n-maghemite type (gamma-Fe₂O₃), n-magnetite (Fe₃O₄) or ferrites of the MeFe₂O₄ type, where Me is a divalent metal selected from manganese, copper, zinc, cobalt, nickel, magnesium, calcium or cadmium, can be used for example as the heating medium. These particles preferably have an average particle size of ≦100 nm, preferably ≦51 nm and in particularly preferably ≦30 nm.

A material that is available from Evonik (formerly Degussa) under the name MagSilica® is suitable, for example. In this material iron oxide crystals measuring 5 to 30 nm are embedded in an amorphous silicic acid matrix. Iron oxide-silicon dioxide composite particles that are described in more detail in the German patent application DE 101 40 089 are particularly suitable.

These particles can contain superparamagnetic iron oxide domains having a diameter of 3 to 20 nm. These are understood to be physically separated superparamagnetic areas. The iron oxide can be present in these domains in a uniform modification or in different modifications. A particularly preferred superparamagnetic iron oxide domain is gamma-Fe₂O₃, Fe₃O₄ and mixtures thereof.

The proportion of superparamagnetic iron oxide domains in these particles can be between 1 and 99.6 wt. %. The individual domains are separated from one another and/or surrounded by a non-magnetizable silicon dioxide matrix. The range having a proportion of superparamagnetic domains of >30 wt. % is preferred, particularly preferably >50 wt. %. As the proportion of superparamagnetic areas increases, so too does the achievable magnetic effect of the particles according to the invention. In addition to physically separating the superparamagnetic iron oxide domains, the silicon dioxide matrix also serves to stabilize the oxidation level of the domains. Thus magnetite for example as the superparamagnetic iron oxide phase is stabilized by a silicon dioxide matrix. These and other properties of these particles that are particularly suitable for the present invention are set out in more detail in DE 101 40 089 and in WO 03/042315.

Furthermore, nanoscale ferrites such as are known for example from WO 03/054102 can be used as the heating medium. These ferrites have a (M^{a}_{1-x-y} M^{b}ₓ Fe^{II}_{y}) Fe^{III}₂O₄ composition, in which

M^{a} is selected from Mn, Co, Ni, Mg, Ca, Cu, Zn, Y and V,
M^{b} is selected from Zn and Cd,
x denotes 0.05 to 0.95, preferably 0.01 to 0.8,
y denotes 0 to 0.95 and
the sum of x and y is at most 1.

It can be provided here that the surface of the solid heating medium is coated with a substance that is catalytically active for at least one of the chemical reactions. If the reactions are performed in a continuous-flow reactor, as described below, different heating media can be provided in succession in the continuous-flow reactor. These can be coated with different catalytically active substances that are specific to the first or to a further chemical reaction respectively.

The method according to the invention is suitable in particular for performing reactions that can be induced thermally. There is in principle no restriction on the possible reaction types, provided that reaction conditions (such as pH for example) are not chosen or starting materials used or products formed that destroy the heating medium. For example, sequences of chemical reactions can be performed in which in at least one of the chemical reactions at least one chemical bond between 2 C atoms or between a C atom and an atom X is formed, broken or rearranged, wherein X can be selected for example from: H, B, O, N, S, P, Si, Ge, Sn, Pb, As, Sb, Bi and halogens. This can also be a rearrangement of chemical bonds such as occurs for example in cycloadditions and Diels-Alder reactions. However, bonds can also be formed and/or dissolved between two identical or different atoms X, wherein X can have the aforementioned meaning. A “formation of a bond” is generally understood here also to mean a conversion of a single bond into a double bond or of a single or double bond into a triple bond, etc. The term “dissolution of a bond” covers the reverse process.

The reaction that can be induced thermally corresponds for example to at least one of the following reaction types: oxidation (including dehydrogenation), reduction (including hydrogenation), fragmentation, addition to a double or triple bond (including cycloaddition and Diels-Alder reaction), substitution (SN1 or SN2, radical), in particular aromatic substitution, elimination, rearrangement, cross-coupling, metathesis reaction, formation of heterocycles, ether formation, ester formation or transesterification, amine or amide formation, urethane formation, pericyclic reaction, Michael addition, condensation, polymerization (radical, anionic, cationic), polymer grafting.

For reduction or hydrogenation reactions the following are suitable as a reducing agent or hydrogen source for example: cycloalkenes such as cyclohexene, alcohols such as ethanol, inorganic hydrogenation reagents such as sodium hydroborate or sodium aluminum hydride.

At least one of the successively proceeding chemical reactions can be an oxidation reaction for example. The following possible embodiments apply here:

One embodiment of the method according to the invention including an oxidation reaction has the characterizing feature that the particles of electrically conductive and/or magnetizable solids contain at least some oxidic groups and serve as oxygen transfer agents for the oxidation reaction. For example, magnetizable metal oxides having an oxidizing effect in respect of the first reactant can be used. In this case the heating medium itself is thus the oxidizing agent (oxygen transfer agent). Examples of this are inductively heatable oxides such as the aforementioned ferrites of the MeFe₂O₄ or (M^{a}_{1-x-y} M^{b}ₓ Fe^{II}_{y}) Fe^{III}₂O₄, gamma-Fe₂O₃, Fe₃O₄ type and mixtures thereof.

An alternative embodiment has the characterizing feature that the heating medium is selected from particles of magnetizable solids and that these are present in the mixture with further particles containing at least some oxidic groups and serving as oxygen transfer agents for the oxidation reaction. These further particles do not themselves have to be directly heatable by electromagnetic induction. They are rather heated and activated by direct (particle-particle contact) or indirect (via the reaction medium) heat transfer from the heatable particles. Oxides of semi-metals and metals for example are suitable here, which can have a plurality of positive oxidation levels if the metal or semi-metal is present in the oxides in a higher oxidation level than the smallest possible positive oxidation level. Examples are: oxides of Ce(IV), Pb(IV), Sb(V), V(V), Cr(IV and higher), Mn(IV and higher), Fe(III), Co(III or IV) and Cu(II). Metal peroxides are likewise suitable, for example selenium peroxide or nickel peroxide, in particular in the form of nanoparticles having an average particle size determined by light scattering methods of less than 100.

In both alternatives the oxidic groups in the particles are depleted as the oxidation reaction progresses, such that the reaction would soon come to a stop. It is therefore preferable to provide that the particles containing at least some oxidic groups and serving as an oxygen transfer agent for the oxidation reaction are provided anew with oxidic groups during or after the oxidation reaction by reaction with oxygen. This means that either oxygen or an oxygen transfer agent is additionally introduced continuously into the reactor such that the oxidic groups of the particles are immediately regenerated again (such that the particles act similarly to a catalyst) or that the particles are regenerated in batches by means of a single or multiple, but interrupted, addition of oxygen or an oxygen transfer agent into the reactor. Examples of oxygen transfer agents are organic peroxides or organic per-acids or anions thereof, inorganic per-acids such as for example peroxosulfuric acid or peroxodisulfuric acid or anions thereof, oxo acids of halogens or anions thereof, such as for example chlorates or perchlorates, or H₂O₂ or compounds capable of cleaving H₂O₂.

A further embodiment of the present invention including an oxidation reaction consists in that the reactor contains no particles containing at least some oxidic groups but rather that the reaction medium itself contains oxygen. This can be achieved for example in that the oxygen is introduced into the reaction medium in dissolved form and/or in the form of the finest possible gas bubbles (for example by feeding it through a diffuser stone). This preferably takes place continuously, such that a continuous reaction control is possible. Oxygen can however also be introduced batchwise. In this embodiment it can be advantageous to perform the oxidation reaction under pressure. Therefore a variant of this embodiment consists in that the reactor is designed as a pressure reactor and the chemical reaction is performed at a pressure above atmospheric pressure, preferably of at least 1.5 bar. Pressures higher than 20 bar should not be necessary in practice but are not excluded.

Where reference is made to “oxygen” within the context of this invention, it can mean pure oxygen or an oxygen-containing gas, in the simplest case air.

A further reactant can however also serve as the oxidizing agent (oxygen transfer agent), which is itself reduced in the oxidation reaction. For this embodiment too, examples of oxygen transfer agents are: organic peroxides or organic per-acids or anions thereof, inorganic per-acids such as for example peroxosulfuric acid or peroxodisulfuric acid or anions thereof, oxo acids of halogens or anions thereof, such as for example chlorates or perchlorates, or H₂O₂ or compounds capable of cleaving H₂O₂.

A further embodiment of the method according to the invention has the characterizing feature that the surface of the solid heating medium is coated with a substance that is catalytically active for the oxidation reaction. This can be a layer of metal oxides, for example, which easily change their oxidation level on absorption and release of oxygen. The aforementioned metal oxides for example are suitable for this purpose. These can be fixed to a polar silica gel layer that encapsulates inductively heatable metal compounds, as is the case for example with the MagSilica™ heating material mentioned above. Alternatively, however, the surface of the heating material is provided with ion-exchange groups such as for example —SO³⁻ or —NR₃⁺, which can bind cations or anions having oxidizing properties by means of ion exchange, for example RuO₄⁻, OsO4²⁻, MnO₄⁻, IO₄⁻, ClO₄⁻, ClO₂⁻, ClO⁻. These oxidizing metal oxides, cations or anions can act stoichiometrically as oxidizing agents. They act as a catalyst if after being reduced by oxygen or oxygen transfer agents in the reaction medium they are converted back into their oxidized form so that they are again available as an oxidizing agent.

Further examples of oxidizing agents are Dess-Martin periodinane or tetramethyl pyridinium oxide. If these are provided with —OH groups, they can be fixed to polar carriers such as for example the silica gel layer of MagSilica™, optionally by means of a linker. The molecular formulae of these compounds are:

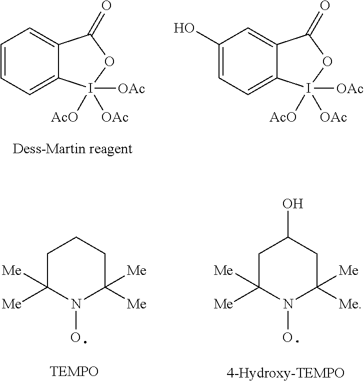

A further embodiment of the method according to the invention has the characterizing feature that in at least one of the chemical reactions a metal-carbon bond is formed or broken. An organometallic compound (i.e. the compound containing a metal-carbon bond) can be a starting product, an intermediate compound or the target compound. The metal to which a carbon atom is bound can be one of the typical metals for organometallic reactions. The following can be cited by way of example: Li, Mg, Al, Sn, Pb, Fe, Co, Ni, Zn, Na, K, B, Si, Cr, Cu, Ce or another metal.

According to this embodiment of the method according to the invention one of the following reactions can be performed as the first or second reaction, the invention not being restricted thereto:
a) Formation of metal alkyls such as for example alkyl magnesium compounds by reacting elemental metals with alkyl halides, including a Reformatsky reaction,
b) Metalation of aromatic compounds by reaction with metal alkyls, for example the lithiation of benzene with alkyl lithium (e.g. butyl lithium),
c) Formation of organometallic compounds by transmetalation

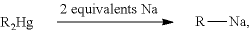

d) Formation of organometallic compounds by metal-halogen exchange

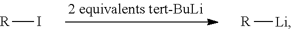

e) Formation of metal alkynes such as for example alkynyl-copper compounds by reacting elemental metals with alkynes, optionally in the presence of a base (e.g. triethylamine),
f) Addition reactions of metal-H, metal-C or metal-metal² (e.g. R₃Si metal) to alkenes and alkynes (hydrometalation, carbometalation, metalometalation),
g) Formation of metal-arene complexes such as for example tricarbonyl(h6-benzene)chromium(0) from benzene and chromium hexacarbonyl,
h) Reaction of organometallic compounds with electrophiles with formation of C—C bonds.

One of the chemical reactions can moreover be a fragmentation. This means that either an intermediate compound or the target compound has a lower molar mass than the starting compound and that in order to produce it from the starting compound at least one covalent bond of the starting compound is cleaved. The covalent bond to be cleaved can in particular be a C—C bond, a C—O bond, a C—N bond, a C—Se bond or a C—S bond.

One of the reactions can for example belong to the following group of reactions, the invention not being restricted thereto.
a) Pyrolysis of oils
b) Depolymerization reactions, for example the cleavage of polyolefins or of cyanoacrylate polymers or oligomers into smaller fragments or monomers,
c) Retro-cycloadditions (such as retro-Diels-Alder reactions),
d) Grob fragmentations (in their general form):

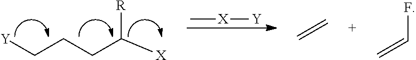

The following embodiments again apply in general and do not require one of the chemical reactions to be one of the aforementioned reactions. However, they apply to these reactions too.

Regardless of whether or not one of the chemical reactions includes a conversion with oxygen, hydrogen or another gas, it can be provided for at least one of the reactions to be performed under pressure. Therefore an embodiment of the method according to the invention consists in general terms in that at least one of the reactors is designed as a pressure reactor and at least one of the chemical reactions is performed at a pressure above atmospheric pressure, preferably of at least 1.5 bar. Pressures higher than 20 bar should not be necessary in practice but are not excluded.

The method according to the invention can in principle be performed continuously or batchwise. If it is performed batchwise, the reaction medium and the inductively heated solid heating medium are preferably moved relative to each other during the reaction. If a particulate heating medium is used, this can take place in particular by stirring the reaction medium together with the heating medium or by vortexing the heating medium in the reaction medium. If for example meshes or wool of a threadlike heating medium are used, the reaction vessel containing the reaction medium and the heating medium can be shaken for example.

A preferred embodiment of a reaction performed batchwise consists of placing the reaction medium in a reaction vessel together with particles of the heating medium and moving it with the aid of a moving element located in the reaction medium, the moving element being set up as an inductor by means of which the particles of the heating medium are heated by electromagnetic induction. Thus in this embodiment the inductor is located inside the reaction medium. The moving element can take the form of a stirrer or a vertically moving plunger, for example.

It can additionally be provided that the reactor is externally cooled during the chemical reaction. This is possible in batch operation in particular, if, as stated above, the inductor is immersed in the reaction medium. The introduction of the electromagnetic alternating field into the reactor is then not inhibited by the cooling device.

The reactor can be cooled internally via cooling coils or heat exchangers or preferably externally. Optionally precooled water or a cooling mixture whose temperature is below 0° C. can be used for cooling, for example. Examples of such cooling mixtures are ice/common salt mixtures, methanol/dry ice or liquid nitrogen. Cooling allows a temperature gradient to be established between the reactor wall and the inductively heated heating medium. This is particularly pronounced if a cooling mixture with a temperature well below 0° C. is used, for example methanol/dry ice or liquid nitrogen. The reaction medium that is heated by the inductively heated heating medium is then cooled again externally. The chemical reaction of the reactant then takes place only if it is in contact with the heating medium or is at least in its immediate vicinity. Owing to the movement of the reaction medium relative to the heating medium, the product species formed in the reaction quickly move into the cooler areas of the reaction medium, thus inhibiting their further thermal reaction. In this way if there are several possible reaction pathways for the reactant(s), a desired reaction pathway can be selected kinetically.

In this batchwise procedure a first chemical reaction can firstly be performed to form an intermediate compound before one or more further reactants is added, with which the intermediate compound formed reacts to form the target compound.

In an alternative embodiment the chemical reactions are performed continuously in continuous-flow reactors arranged in series, each continuous-flow reactor being at least partially filled with the solid heating medium and thus having at least one heating zone that is capable of being heated by electromagnetic induction, the reaction medium flowing continuously through the continuous-flow reactor and the inductor being located outside the reactor. Here the reaction medium flows around the heating medium, if for example the latter is in the form of particles, chips, wires, meshes, wool, fillers, etc. Alternatively, the reaction medium flows through the heating medium through a large number of holes in the heating medium if the latter consists for example of one or more membranes, frits, porous fillers or a foam.

Each continuous-flow reactor is preferably designed as a tubular-flow reactor. In this case the inductor can surround the reactor completely or at least partially. The electromagnetic alternating field generated by the inductor is then introduced on all sides or at least from several points into the reactor.

“Continuous” is understood here in the usual way to be a reaction control in which the reaction medium flows through the reactor at least over a period such that a total volume of reaction medium that is large in comparison to the internal volume of the reactor itself has flowed through the reactor before the flow of the reaction medium is stopped. “Large” in this sense means “at least twice as large”. Even such a continuously performed reaction has a beginning and an end of course.

The heating medium can be the same in both reactors. However, depending on the reaction type and the reaction control requirements, the heating media in the two reactors can also be different. Similarly, the inductors for the two reactors can be of identical or different design and can be operated at the same or different outputs. Correspondingly, the temperatures in the two reactors can be the same or different. Ideally the reaction conditions in the two reactors are adapted to each other such that the product of the first reaction in the first reactor is transferred from the first reactor to the second reactor in a quantity such that it is available for a further reaction in the second reactor.

The two reactors can be separated from each other by a connecting element, through which the reaction medium flows from the first to the second reactor. However, the two reactors can also open directly into each other, the boundary between the first and the second reactor being defined by the fact that the reaction conditions (temperature, type of heating medium, catalyst, addition of a further reactant) change at said boundary. For example, different heating zones can be heated to differing extents, resulting in a “first reactor” and a “second reactor”. This can be achieved either by the positioning of different heating media in the continuous-flow reactor or by means of differently designed inductors along the reactor. Different heating conditions can be established in this way for the formation of the intermediate compound and the target compound.

A cooling zone can be provided after the (last) heating zone if desired, for example in the form of a cooling jacket around the reactor.

It can also be provided that after leaving the heating zone of the second reactor the reaction medium is brought into contact with an absorber substance that removes by-products or impurities from the reaction medium. This can for example be a molecular sieve, through which the reaction medium flows after leaving the heating zone. This allows the product to be purified immediately after being produced.

Depending on the speed of the chemical reaction, the product yield can optionally be increased by returning at least part of the reaction medium that has flowed through the solid heating medium in one of the reactors to flow through the solid heating medium in this reactor again. It can be provided for impurities, by-products or the desired main product to be removed from the reaction medium after each passage through the solid heating medium. The various known separating methods are suitable for this purpose, for example absorption on an absorber substance, separation by a membrane method, precipitation by cooling or separation by distillation. A complete reaction of the reactant(s) can ultimately be achieved in this way.

The total contact time of the reaction medium with the individual inductively heated heating medium that is conveniently chosen is dependent on the kinetics of the individual chemical reaction. The slower the desired chemical reaction, the longer the chosen contact time should be. This must be adjusted empirically in the individual case and can be done by adapting the flow rate, the diameter of the free reactor (i.e. not containing the heating medium) and the reactor length for each reactor. As a reference point, the reaction medium should preferably pass through the continuous-flow reactor one or more times at a rate such that the total contact time of the reaction medium with the inductively heated heating medium in each reactor is in the range from approximately 1 second to approximately 2 hours before the target compound is separated off. Shorter contact times are conceivable, but are more difficult to control. Longer contact times can be necessary with particularly slow chemical reactions, but they incrementally reduce the cost-effectiveness of the method.

The method according to the invention is preferably performed in such a way that under the established reaction conditions (in particular temperature and pressure) the reaction medium is present in the corresponding reactor as a liquid. Relative to the reactor volume, better volume/time yields are generally possible in this way than is the case with reactions in the gas phase.

It goes without saying that the nature of the heating media and the design of the inductors must be matched to one another so that the reaction medium can be heated in the desired way. Critical variables here are the output of the inductor expressed in Watts and the frequency of the alternating field generated by the inductor. In principle, the greater the mass of the heating medium to be heated inductively, the higher the chosen output must be. In practice, the achievable output is limited in particular by the possibility of cooling the generator needed to supply the inductor.

Inductors generating an alternating field with a frequency in the range from approximately 1 to approximately 100 kHz, preferably from 10 to 80 kHz and in particular in the range from approximately 10 to approximately 50 kHz, particularly up to 30 kHz, are particularly suitable. Such inductors and the associated generators are available commercially, for example from IFF GmbH in Ismaning, Germany.

The inductive heating is thus preferably performed with an alternating field in the medium-frequency range. In comparison with an excitation at higher frequencies, for example at those in the high-frequency range (frequencies above 0.5, in particular above 1 MHz), this has the advantage that the introduction of energy into the heating medium is more readily controllable. This applies in particular if the reaction medium is present as a liquid under the reaction conditions. In the context of the present invention it is therefore preferable for the reaction medium to be present as a liquid and for inductors to be used that generate an alternating field in the aforementioned medium-frequency range. This allows a cost-effective and readily controllable reaction control.

The following for example can be used as the heating medium:
a) MagSilica® 58/85 from Evonik (formerly Degussa),
b) Manganese ferrite powder,
c) Bayferrox® 318 M: synthetic alpha-Fe₃O₄ from Harold Scholz & Co. GmbH,
d) Manganese zinc ferrite, surface-coated with oleic acid, ferrite content 51.7 wt. %,
e) Balls or other moldings, rolled metal sheets, chips, rolled meshes or metal wool,
f) Fe₂O₃, in particular in the form of nanoparticles having a particle size in the range from 20 to 200 nm, or Fe₃O₄, in particular in the form of nanoparticles having a particle size in the range from 20 to 200 nm (both available from DKSH GmbH, Germany),
g) Steel balls, for example ball bearings, preferably having a diameter of at most 1 mm, for example between 0.5 and 1 mm.

In a special embodiment of the method according to the invention the heating medium is ferromagnetic and has a Curie point in the range from approximately 40 to approximately 250° C., which is selected such that the Curie point differs by no more than 20° C., preferably by no more than 10° C., from the selected reaction temperature. This leads to an inherent protection against inadvertent overheating. The heating medium can only be heated by electromagnetic induction up to its Curie point, while at a higher temperature it is no longer heated by the electromagnetic alternating field. Even if the inductor malfunctions, the temperature of the reaction medium can be prevented in this way from rising inadvertently to a value well above the Curie point of the heating medium. If the temperature of the heating medium falls below its Curie point again, it can once again be heated by electromagnetic induction. This leads to a self-regulation of the temperature of the heating medium in the vicinity of the Curie point.

Glass tubes or PEEK reactors of length 10 to 20 cm, for example of length 12 cm, and of various internal and external diameters can be used for example as tubular-flow reactors. The tubes can be provided with screw connections at both ends to allow HPLC attachments and the appropriate hoses to be attached.

A suitable inductor has the following performance features: inductance: 134 μHenry, number of coil turns: 2·16, cross-sectional area=2.8 mm² (the cross-sectional area is calculated from the number of conductor wires used in the inductor and their diameter). The diameter of the gap to hold the tubular-flow reactors can be 12 mm. The inductor is operated at a frequency of 25 kHz, for example.

### EMBODIMENT EXAMPLE

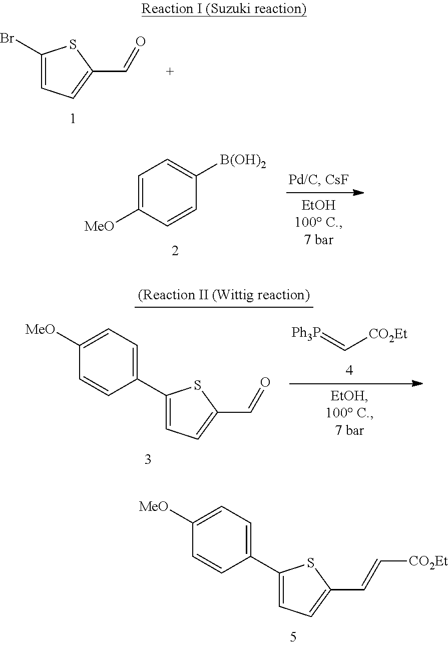

A PEEK reactor (length 12 cm, internal diameter 8.5 mm) is filled with MagSilica™ and a palladium-on-carbon catalyst and both ends are closed with cotton wool. One end of the reactor is connected to an HPLC pump, while the other end is connected via a T-mixer and a pressure valve (7 bar) to a second PEEK reactor (length 12 cm, internal diameter 8.5 mm). The second branch of the T-mixer is connected to a second HPLC pump. The second PEEK reactor is filled with MagSilica™. The other end of the PEEK reactor is connected to a pressure valve (7 bar), which ends in a receiver. Both reactors are placed in inductors and the entire system is flushed with ethanol. A temperature of 100° C. is established at both inductors (excitation frequency 250 per mil in each case). A flow rate of 0.1 ml/min is set at both HPLC pumps. Once a constant flow rate and temperature have been established, a solution of aryl bromide 1, boronic acid 2 and cesium fluoride in ethanol (5 ml) is pumped through the system with the first HPLC pump. A solution of Wittig reagent 4 in ethanol is introduced into the T-mixer using the second HPLC pump. There the reaction solution is mixed with the product 3 of the first reaction and is reacted in the second reactor. A further 15 ml of ethanol are then pumped through the system by the first and second HPLC pumps respectively. The combined organic phases are concentrated to low volume under vacuum and the residue is washed repeatedly with water. The aqueous phase is extracted twice with ethyl acetate and the combined organic phases are dried over MgSO₄. After concentrating the organic phase to low volume the residue is purified by column chromatography (SiO₂, ethyl acetate/petroleum ether 50:1) and the target compound 5 is obtained in a yield of 80%.

1. A method for performing at least two successive chemical reactions to produce a target compound from at least one first, and at least one second and/or further reactant, encompassing a first reaction and a further reaction in a reactor containing a reaction medium, the reaction medium in the reactor being brought into contact with a solid heating medium that can be heated by electromagnetic induction and that is located inside the reactor and surrounded by the reaction medium, and the heating medium being heated by electromagnetic induction with the aid of an inductor, an intermediate compound being formed in a first reaction, from which the target compound forms in a further reaction, and at least one reactant that was not present in the reactor before the first reaction being added to the reactor between the first and the further reaction, and the target compound being separated from the heating medium.

2. The method according to claim 1, through which the target compound forms by one of the following reaction sequences:
a) within the first reactant an intramolecular bond rearrangement or a fragmentation takes place as a first reaction to form an intermediate compound, which is reacted with one or more second reactants in a further reaction to form the target compound;
b) the first reactant reacts in a first reaction with a second reactant to form an intermediate compound, from which the target compound forms in a further reaction with a further reactant;
c) the first reactant reacts in a first reaction with a second reactant to form a first intermediate compound, from which in a second reaction a second intermediate compound forms through an intramolecular bond rearrangement or a fragmentation, from which the target compound forms in a further reaction with a further reactant.

3. The method according to claim 1, the first reaction taking place in a first reactor and the further reaction taking place in a second reactor, each containing a reaction medium, the reaction medium in each reactor being brought into contact with a solid heating medium that can be heated by electromagnetic induction and that is located inside the reactor and surrounded by the reaction medium, and the heating medium being heated by electromagnetic induction with the aid of an inductor, at least one reactant being added to the second reactor which was not present in the first reactor.

4. The method according to claim 1, wherein the heating medium is selected from particles of electrically conductive and/or magnetizable solids, the particles having an average particle size in the range from 1 to 1000 nm.

5. The method according to one claim 1, wherein the heating medium is selected from particles of electrically conductive and/or magnetizable solids, each particle containing at least one core of an electrically conductive and/or magnetizable material which is sheathed by a non-magnetic material.

6. The method according to claim 1, wherein at least one reactor is designed as a pressure reactor and at least one of the chemical reactions is performed at a pressure above atmospheric pressure, preferably of at least 1.5 bar.

7. The method according to claim 1, wherein the surface of at least one solid heating medium is covered with a substance that has a catalytic activity for at least one of the chemical reactions.

8. The method according to claim 1, wherein the chemical reactions are each performed in a continuous-flow reactor that is at least partly filled with the solid heating medium and thus has at least one heating zone that can be heated by electromagnetic induction, the reaction medium flowing through the continuous-flow reactor and the inductor being located outside the reactor.

9. The method according to claim 8, wherein the reaction medium flows through each continuous-flow reactor at a speed such that the contact time between the reaction medium and the heating medium in each reactor is in the range from one second to two hours.

10. The method according to claim 1, wherein the reaction medium is present in each reactor as a liquid.

11. The method according to claim 1, wherein each inductor generates an alternating field having a frequency in the range from 1 to 100 kHz, preferably in the range from 10 to 80 kHz and in particular up to 50 kHz.
